(19) United States
(12) Patent Application Publication
(10) Pub. No.: US 2022/0276150 A1
(43) Pub. Date: Sep. 1, 2022

(54) Optoacoustic Probe

(71) Applicant: Nippon Telegraph and Telephone Corporation, Tokyo (JP)

(72) Inventors: Yujiro Tanaka, Tokyo (JP); Takuro Tajima, Tokyo (JP)

(73) Assignees: Nippon Telegraph and Telephone Corporation, Tokyo (JP); Nippon Telegraph and Telephone Corporation, Tokyo (JP)

(21) Appl. No.: 17/637,602

(22) PCT Filed: Sep. 11, 2019
(86) PCT No.: PCT/JP2019/035701

§ 371 Date: Feb. 23, 2022

(51) Int. Cl. G01N 21/17; G02F 1/33; G02F 1/31; G02B 26/08

(52) U.S. Cl. CPC G01N 21/1702; G02F 1/33; G02F 1/31; G02B 26/0816; G02F 2203/02; G02F 2201/02; G01N 2201/0636; A61B 5/0095

(57) ABSTRACT

A photoacoustic probe includes a light source that emits light, an acoustic sensor that is arranged such that an axial direction is in parallel to a depth direction of an object to be measured and detects a sound produced from the object to be measured, a propagation member that propagates the light from the light source to the object to be measured, and propagates the sound produced from the object to be measured by emission of the light from the light source to the acoustic sensor, a reflection member that is provided within the propagation member, reflects the light from the light source, and emits reflected light to the object to be measured in the axial direction of the acoustic sensor, and a sweep mechanism capable of changing a position at which the light from the light source enters the reflection member.

This patent application is a national phase filing under section 371 of PCT application no. PCT/JP2019/035701, filed on Sep. 11, 2019, which application is hereby incorporated herein by reference.

### TECHNICAL FIELD

The present invention relates to a photoacoustic probe to be used in an imaging apparatus that visualizes an optical absorption coefficient distribution of an object to be measured, a component concentration measurement apparatus that measures the concentration of a specific component contained in an object to be measured, and the like.

### BACKGROUND

Spatial information about an interstitial fluid component such as sugar, blood vessels, and the like is effective for early detection of diabetes or malignant neoplasm. A photoacoustic method is a method of finding an optical absorption property of a substance, utilizing the fact that, when emitting light to the substance, acoustic waves are produced by local thermal expansion in accordance with an absorption wavelength range of the substance (see Patent Literature 1). In addition, acoustic waves produced in the photoacoustic method are a type of ultrasonic waves, have a wavelength longer than light, and are thus unlikely to be affected by scattering of an object to be measured. Therefore, the photoacoustic method is receiving attention as a technique of visualizing an optical absorption property in an object to be measured having significant scattering, such as a living body.

A technique of scanning an object to be measured with an optical spot at which excitation light is collected and detecting ultrasonic waves produced at each position of the object to be measured with an acoustic sensor or the like is being used. Since, in the case of scanning the object to be measured with the optical spot, ultrasonic waves are produced when an absorbing substance is present in the object to be measured, the optical absorption property of the object to be measured can be visualized by detecting the ultrasonic waves. Alternatively, there is also a technique of uniformly emitting excitation light to an object to be measured, and based on the time until an acoustic sensor receives ultrasonic waves after the excitation light is emitted, estimating a position at which a light absorbing substance absorbs light and produces ultrasonic waves. Alternatively, by moving an object to be measured to change the position on the object to be measured to which an optical spot is emitted, the object to be measured is scanned.

However, the conventional methods require an acoustic sensor to be brought into contact with an object to be measured in order to acquire ultrasonic waves, thereby forming a path (acoustic matching layer) through which the ultrasonic waves propagate, which raises a problem in that usage scenes are restricted. It is difficult to mount only an interface unit 101 of an imaging apparatus 100 on an arm of a living body 102, for example, as illustrated in FIG. 11 to measure an optical absorption coefficient distribution. That is, in conventional apparatuses, a measurement site is a single point, and in order to obtain the optical absorption coefficient distribution of an object to be measured, relative positions of an interface unit on which an acoustic sensor is mounted and the object to be measured need to be changed to perform measurement many times. Thus, measurement takes time, and further, the contact state between the interface unit and the object to be measured changes, which raises a problem in that accurate data cannot be obtained.

### CITATION LIST

### Patent Literature

Patent Literature 1: Japanese Patent Laid-Open No. 2018-79125.

### SUMMARY

### Technical Problem

Embodiments of the present invention were made to solve the above problems, and have an objective to provide a photoacoustic probe that can scan an object to be measured with an optical spot without changing the contact state with the object to be measured.

### Means for Solving the Problem

A photoacoustic probe of embodiments of the present invention includes: a light source configured to emit one or more light rays; an acoustic sensor arranged such that an axial direction is in parallel to a depth direction of an object to be measured, and configured to detect a sound produced from the object to be measured; a propagation member configured to propagate light from the light source to the object to be measured, and propagate the sound produced from the object to be measured by emission of the light from the light source to the acoustic sensor; a reflection member provided within the propagation member, and configured to reflect the light from the light source and emit reflected light to the object to be measured in the axial direction of the acoustic sensor; and a sweep mechanism capable of changing a position at which the light from the light source enters the reflection member.

### Effects of Embodiments of the Invention

According to embodiments of the present invention, an object to be measured can be scanned with an optical spot without changing the contact state with the object to be measured. In embodiments of the present invention, it is not necessary to change the relative positions of a photoacoustic probe and the object to be measured, and the time required for measuring an optical absorption coefficient distribution of the object to be measured and measuring a component concentration can be shortened. In addition, in embodiments of the present invention, the contact state between the photoacoustic probe and the object to be measured is not changed during measurement, which can improve the measurement accuracy.

### BRIEF DESCRIPTION OF THE DRAWINGS

FIG. 1 is a block diagram illustrating a configuration of an imaging apparatus according to an embodiment of the present invention.

FIG. 2 is a drawing illustrating time changes in sound pressure detected by an acoustic sensor.

FIG. 3 is a drawing illustrating an example of a sweep mechanism of the imaging apparatus according to an embodiment of the present invention.

FIG. 4 is a drawing illustrating another example of the sweep mechanism of the imaging apparatus according to an embodiment of the present invention.

FIG. 5 is a drawing illustrating another example of the sweep mechanism of the imaging apparatus according to an embodiment of the present invention.

FIG. 6 is a drawing illustrating another example of the sweep mechanism of the imaging apparatus according to an embodiment of the present invention.

FIG. 7 is a drawing illustrating another example of the sweep mechanism of the imaging apparatus according to an embodiment of the present invention.

FIG. 8 is a drawing illustrating a relation between the frequency of ultrasonic waves produced from an object to be measured and the radius of an optical spot.

FIG. 9 is a drawing illustrating an example of a columnar optical spot formed in an object to be measured.

FIG. 10 is a block diagram illustrating a configuration example of a computer that achieves the imaging apparatus according to an embodiment of the present invention.

FIG. 11 is a drawing illustrating an example of a measurement form in a conventional photoacoustic method.

### DETAILED DESCRIPTION OF ILLUSTRATIVE EMBODIMENTS

### Principles of Embodiments of the Invention

An acoustic sensor that converts acoustic waves produced from an object to be measured into an electric signal is designed so as to have the highest detection sensitivity in a case in which plane waves enter vertically. Embodiments of the present invention use a mechanism of selectively guiding light to an object to be measured directly under an acoustic sensor such that a sound source is positioned directly under the acoustic sensor, and selectively guiding acoustic waves from the object to be measured to the acoustic sensor without interference. Accordingly, the object to be measured is scanned with an optical spot without changing the contact state between an interface unit of an apparatus and the object to be measured, and information about a three-dimensional optical absorption property of the object to be measured is acquired.

In addition, the frequency of the acoustic waves produced from the object to be measured changes according to the optical spot. In a case in which an interstitial fluid component having a small light absorption contrast or the like is a target of measurement, it is necessary to emit light to a wider region in order to average the distribution of tissues as compared to a case in which the target of measurement is blood cells or the like. When light is emitted to a wide region, the frequency of ultrasonic waves produced is approximately 1 MHz, which is a lower frequency than that of ultrasonic waves of several megahertz to several hundreds of megahertz produced in the case in which the target of measurement is blood cells or the like. As a result, a sound collection effect produced by an acoustic lens or the like is extremely reduced. Thus, in embodiments of the present invention, the size of an optical spot to be formed within an object to be measured is set such that the frequency of acoustic waves produced from the object to be measured has a desired value (a sensitivity bandwidth of an acoustic sensor), thereby achieving highly sensitive measurement.

### Embodiment

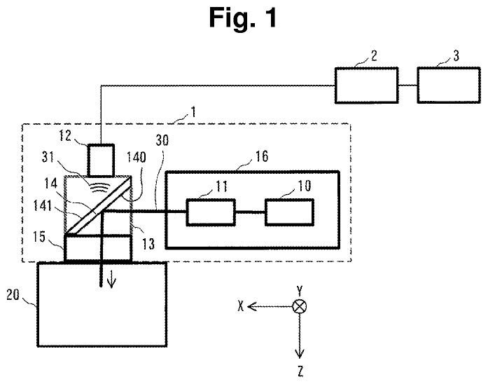

Hereinafter, embodiments of the present invention will be described with reference to the drawings. FIG. 1 is a block diagram illustrating a configuration of an imaging apparatus according to an embodiment of the present invention. The imaging apparatus includes a photoacoustic probe 1, a calculation unit 2 that calculates an optical absorption coefficient distribution of an object to be measured 20 based on a sound received by the photoacoustic probe 1, and a recording unit 3 that stores a calculation result obtained by the calculation unit 2.

The photoacoustic probe 1 includes a light source 10 that emits one or more light rays, an optical system 11 that performs collection and beam shaping of light from the light source 10, an acoustic sensor 12 that receives a sound produced from the object to be measured 20 by the photoacoustic effect for conversion into an electric signal that is in proportion to a sound pressure, a propagation member 13 that propagates light from the optical system 11 to the object to be measured 20, and propagates the sound produced from the object to be measured 20 to the acoustic sensor 12, a reflection member 14 that is provided within the propagation member 13, reflects the light from the optical system 11, and emits reflected light to the object to be measured 20 in the axial direction of the acoustic sensor 12, a light-transmissive acoustic matching layer 15 provided between the propagation member 13 and the object to be measured 20, and a sweep mechanism 16 that scans the object to be measured 20 with an optical spot by changing the position at which the light from the optical system 11 enters the reflection member 14.

In the present embodiment, directions parallel to the surface of the object to be measured 20 are denoted by an X direction and a Y direction, and a depth direction of the object to be measured 20 is denoted by a Z direction.

As illustrated in FIG. 1, the photoacoustic probe 1 is placed such that the acoustic matching layer 15 is in contact with a surface of the object to be measured 20, and the axial direction of the acoustic sensor 12 (the direction in which the sensitivity is the highest) is substantially parallel to the depth direction of the object to be measured 20.

A light emitting element such as a laser diode, for example, can be used as the light source 10 of the photoacoustic probe 1. The optical system 11 will be described later. Examples of the acoustic sensor 12 include a microphone through use of a piezoelectric sensor.

As the material of the propagation member 13, a material having high transmittance with respect to utilized light can be used. In the visible light region to the near-infrared light region, examples of the material of the propagation member 13 include a light-transmissive plastic, a light-transmissive glass, a light-transmissive rubber, water, and the like as in Table 1, for example. Note that, in a case of using water, a hollow member formed of a light-transmissive plastic or the like, for example, needs to be filled with water.

TABLE 1

| | Sound | Acoustic | |
|---|---|---|---|
| | speed | impedance | Thickness allowed for |
| Material | (km/sec) | (MRayl) | reflection part (μm) |
| Plastics | 1.9 to 2.7 | 1.7 to 3.2 | 190 to 270 |
| Glasses | 5 | 11 to 20 | 510 |
| Rubbers | 1 to 2 | 1 to 4 | 100 to 200 |
| Water | 1.5 | 1.5 | 150 |

As the reflection member 14, a metal or dielectric film can be used. In a case of causing excitation light 30 to enter the propagation member 13 from the optical system 11 in a direction (the X direction in FIG. 1) vertical to the axial direction of the acoustic sensor 12, the reflection member 14 is a planar metal or dielectric film arranged within the propagation member 13 so as to have an angle of 45 degrees with respect to the X direction and the Z direction. By moving the position at which the excitation light 30 enters the reflection member 14 in the X, Y, and Z directions with the sweep mechanism 16, the position of the optical spot within the object to be measured 20 can be moved.

In addition, it is desirable that the reflection member 14 be sufficiently thinner than the wavelength of ultrasonic waves 31 so as not to interfere with propagation of the ultrasonic waves 31 produced from the object to be measured 20 by the photoacoustic effect, and being less than or equal to approximately 1/10 is desirable.

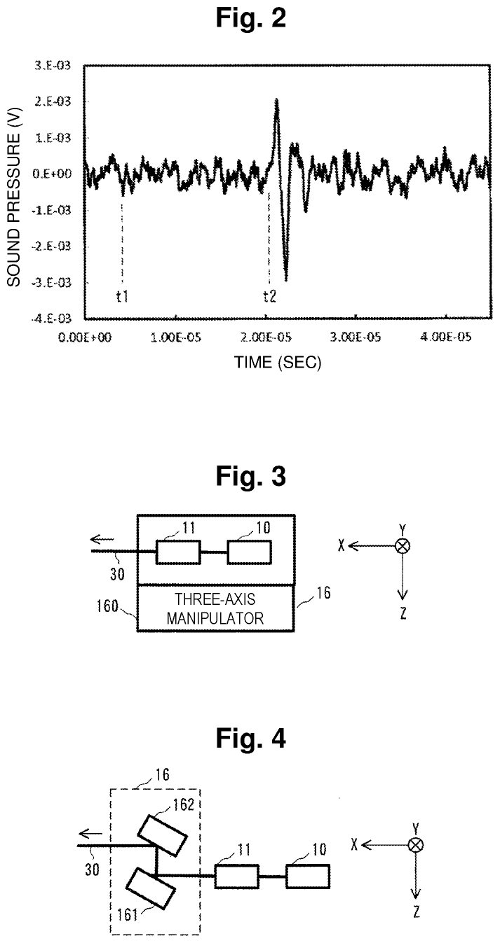

FIG. 2 is a drawing illustrating time changes in sound pressure detected by the acoustic sensor 12. In FIG. 2, t1 denotes a time at which light is emitted from the light source 10, and t2 denotes a time at which the ultrasonic waves 31 are received by the acoustic sensor 12. A sound wavelength λ can generally be expressed as follows from the relation between a sound speed c and a frequency f.

Expression 1:

λ=c/f (1)

Materials that can be considered to be used as the material of the propagation member 13 in terms of acoustic properties and optical properties are as described above. The thickness allowed for the reflection member 14 has values shown in Table 1 with respect to various materials used for the propagation member 13 in a case in which the central frequency of the ultrasonic waves 31 is 1 MHz, and the object to be measured 20 is a living body. In a case of using a metal or dielectric film as the reflection member 14, a thickness of approximately several hundreds of nanometers is generally sufficient. Even in a case in which the frequency of the ultrasonic waves 31 is as high as 100 MHz, the wavelength of the ultrasonic waves 31 is approximately 15 m, so that the reflection member 14 is sufficiently thin, and does not interfere with propagation of the ultrasonic waves 31.

The excitation light 30 does not enter a surface 141 opposite to a reflection surface 140 of the reflection member 14. If an acoustic matching layer (not shown) is formed on this surface 141 where the excitation light 30 does not enter, the optical property that affects scanning with the excitation light 30 is not changed, which is desirable.

The size of the propagation member 13 needs to be designed taking the focal length of the optical system 11 and the refractive index of the material of the propagation member 13 into consideration. Specifically, the size of the propagation member 13 needs to be designed so as to have a sufficient operating distance (a sufficient area of the reflection member 14) such that an optical path length to the position of the optical spot within the object to be measured 20 can be ensured, and a wide range of the object to be measured 20 can be scanned with the optical spot.

It is desirable that light reflected at every point on the reflection surface 140 of the reflection member 14 have an equal optical path length from the perspective of equalizing optical loss. The cross section of the propagation member 13 is considered to have a shape such as a square.

In a case of using collimated parallel light as the excitation light 30, it is not necessary to scan the object to be measured 20 in the depth direction. A position DZ in the depth direction of an absorbing substance within the object to be measured 20 (the distance from the surface of the object to be measured 20 to the absorbing substance) may be estimated by the following expression based on a known sound speed v1 within the object to be measured 20, a known sound speed v2 within the propagation member 13, a known sound path length SL within the propagation member 13, a time t1 at which light is emitted from the light source 10, and a time t2 at which the ultrasonic waves 31 are received by the acoustic sensor 12.

Expression 2:

DZ=v1×{t2−t1−SL/v2} (2)

In general, in a case in which a sound propagates from a first medium to a second medium, an energy transmittance T of the sound is expressed by the following expression where Z1 denotes the acoustic impedance of the first medium, and Z2 denotes the acoustic impedance of the second medium.

Expression 3:

T=(4×Z2)/(Z1+Z2)² (3)

In the example of FIG. 1, the object to be measured 20 is the first medium, and the propagation member 13 is the second medium. In order to efficiently acquire the ultrasonic waves 31 from the object to be measured 20, it is desirable to provide the acoustic matching layer 15 between the object to be measured 20 and the propagation member 13. In a case in which a third medium having an acoustic impedance ZM is inserted between the first medium and the second medium described above, the energy transmittance T of the sound can be expressed by the following expression.

Expression 4:

T=4×(Z1/ZM)×(1+tan(A)²)/((Z1/Z2+1)²+(Z1/ZM+ZM/Z2)²×tan(A)²) (4)

Expression 5:

A=2π×L/X (5)

In Expression (5), L denotes the thickness of the third medium in the Z direction. The acoustic impedance ZM and the thickness L required from the third medium in order to maximize the energy transmittance T are as follows.

Expression 6:

ZM=(Z1×Z2)^{0.5} (6)

Expression 7:

L=¼λ (7)

In a case of using glass, for example, as the acoustic matching layer 15 which is the third medium, a material whose acoustic impedance ZM is approximately 4 MRayl may be used. A plurality of the acoustic matching layers 15 may be provided in a manner overlapping each other in the Z direction. Specifically, in a case in which a material such as glass having a great difference in acoustic impedance from a living body is used as the acoustic matching layer 15, a plurality of the acoustic matching layers 15 may be used such that matching can be achieved between the living body and the propagation member 13 in a stepwise manner within a range in which optical properties such as loss of excitation light are not affected.

As described above, the sweep mechanism 16 moves the position at which the excitation light 30 enters the reflection member 14 in the X, Y, and Z directions. The position of the optical spot within the object to be measured 20 can thereby be changed. In particular, in a case of collecting light by the optical system 11, the position of the optical spot at which light is collected in the object to be measured 20 can be changed by moving the optical system 11 in the X direction. In the present embodiment, the position to which the excitation light 30 is emitted can be changed without changing the contact state between the object to be measured 20 and the photoacoustic probe 1, so that the object to be measured 20 can be scanned with the optical spot, and a three-dimensional optical absorption coefficient distribution of the object to be measured 20 can be obtained.

FIG. 3 to FIG. 7 are drawings illustrating examples of the sweep mechanism 16. The sweep mechanism 16 in FIG. 3 includes a three-axis manipulator 16o that can move the light source 10 and the optical system 11 in the X, Y, and Z directions.

The sweep mechanism 16 in FIG. 4 includes a mirror 161 that can pivot about an axis in the X direction, for example, and a mirror 162 that can pivot about an axis in the Y direction, for example. The mirror 161 reflects the excitation light 30 from the optical system 11. The mirror 162 further reflects the excitation light 30 reflected by the mirror 161 to cause the excitation light 30 to enter the reflection member 14. By causing each of the mirrors 161 and 162 to pivot, the position at which the excitation light 30 enters the reflection member 14 can be changed, and the position of the optical spot within the object to be measured 20 can be changed.

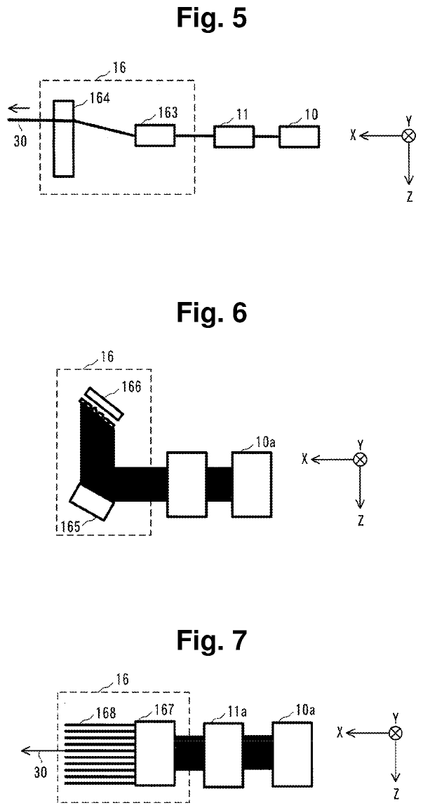

The sweep mechanism 16 in FIG. 5 includes an acousto-optic modulator (AOM) 163 and a converging lens 164. The AOM 163 deflects the excitation light 30 from the optical system 11. The converging lens 164 converges the excitation light 30 from the AOM 163 to cause the excitation light 30 to enter the reflection member 14. By changing the deflection angle of the excitation light 30 with the AOM 163, the position at which the excitation light 30 enters the reflection member 14 can be changed, and the position of the optical spot within the object to be measured 20 can be changed.

The sweep mechanism 16 in FIG. 6 includes a mirror 165 that reflects light from an optical system 11a, and a mirror array device 166 that selectively causes specific light rays among light rays reflected by the mirror 165 to enter the reflection member 14. In the example of FIG. 6, an array light source 10a in which light emitting elements such as laser diodes, for example, are arranged two-dimensionally along the Y-Z plane is used as a light source. The optical system 11a collects respective light rays from the plurality of light emitting elements of the array light source 10a for conversion into a plurality of parallel light rays. The mirror 165 reflects the plurality of parallel light rays from the optical system 11a.

As the mirror array device 166, there are a polygon mirror in which a plurality of reflection surfaces provided parallel to or at an inclination from a rotational axis are arranged two-dimensionally, and a MEMS (Micro Electro Mechanical Systems) mirror array device in which a plurality of micro-mirrors arranged two-dimensionally can pivot independently. In a case of using a polygon mirror as the mirror array device 166, it is possible to, by causing the polygon mirror to pivot, cause only specific parallel light rays among a plurality of parallel light rays reflected by the mirror 165 to enter the reflection member 14 as the excitation light 30, and prevent the remaining parallel light rays from entering the reflection member 14. In a case of using a MEMS mirror array device as the mirror array device 166, it is possible to, by causing a plurality of micro-mirrors to pivot independently, cause only specific parallel light rays among a plurality of parallel light rays reflected by the mirror 165 to enter the reflection member 14 as the excitation light 30, and prevent the remaining parallel light rays from entering the reflection member 14. In this manner, the position at which the excitation light 30 enters the reflection member 14 can be changed.

The sweep mechanism 16 in FIG. 7 includes an optical switch 167 that selectively passes specific light rays among light rays from the optical system 11a, and a fiber bundle 168 obtained by binding a plurality of optical fibers. Also in the example of FIG. 7, the array light source 10a is used as a light source. The optical switch 167 passes only specific parallel light rays among a plurality of parallel light rays from the optical system 11a, and interrupts the remaining parallel light rays. Only the specific parallel light rays having passed through the optical switch 167 enter the fiber bundle 168, and light output from the fiber bundle 168 enters the reflection member 14 as the excitation light 30. By switching selection of light with the optical switch 167, the position at which the excitation light 30 enters the reflection member 14 can be changed, and the position of the optical spot within the object to be measured 20 can be changed.

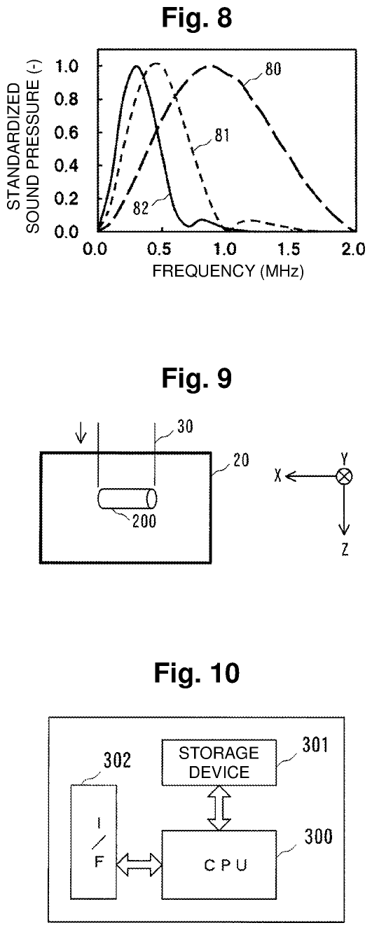

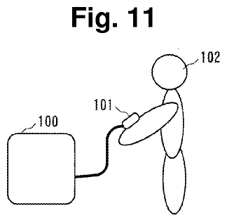

FIG. 8 illustrates a relation between the frequency of the ultrasonic waves 31 produced from the object to be measured 20 by the photoacoustic effect and the radius of an optical spot. In FIG. 8, reference number 80 indicates the frequency of the ultrasonic waves 31 in a case in which the radius of the optical spot within the object to be measured 20 is 0.5 mm, reference number 81 indicates the frequency of the ultrasonic waves 31 in a case in which the radius of the optical spot is 1.0 mm, and reference number 82 indicates the frequency of the ultrasonic waves 31 in a case in which the radius of the optical spot is 1.5 mm. In this manner, the frequency of the ultrasonic waves 31 changes according to the size of the optical spot.

Thus, in a case in which a relatively large tissue having an approximately millimeter-order spatial extent within the object to be measured 20 is a target of measurement, by adjusting the size of the optical spot with the optical system 11/11a, the ultrasonic waves 31 produced from the object to be measured 20 can be adjusted to have a frequency at which the acoustic sensor 12 has a high sensitivity, or to have a frequency less than or equal to 1 MHz at which the propagation efficiency is excellent. The beam waist size, depth of focus, or the like may be adjusted to adjust the size of the optical spot. The optical systems 11, 11a that enable such adjustment can be achieved by a combination of common optical elements such as a beam expander, a convex lens, a concave lens, and the like.

Alternatively, in a case in which subjects uniformly distributed within the object to be measured 20 are the target of measurement, not only the size but also the shape of the optical spot can be changed arbitrarily. When an optical spot 200 formed within the object to be measured 20 as illustrated in FIG. 9, for example, has a columnar shape whose circular cross section is parallel to the Z direction, and the radius of the column is adjusted, the ultrasonic waves 31 produced from the object to be measured 20 can be adjusted to have a frequency at which the acoustic sensor 12 has a high sensitivity, or to have a frequency at which the propagation efficiency is excellent, which can increase the measurement sensitivity. The optical systems 11, 11a that enable such adjustment can be achieved by a combination of common optical elements such as a beam expander, a convex lens, a concave lens, a cylindrical lens, an anamorphic lens, a prism, and the like.

The calculation unit 2 controls the sweep mechanism 16. In addition, the calculation unit 2 can calculate the optical absorption coefficient of the object to be measured 20 based on a sound received by the acoustic sensor 12. As described above, since the object to be measured 20 is scanned with the optical spot, the optical absorption coefficient distribution of the object to be measured 20 can be obtained. The recording unit 3 stores a calculation result obtained by the calculation unit 2.

In addition, the present embodiment has been described using the example in which the photoacoustic probe 1 is applied to an imaging apparatus, but the photoacoustic probe 1 may be applied to a component concentration measurement apparatus. In this case, the calculation unit 2 calculates the concentration of a component as a target of measurement contained in the object to be measured 20 based on at least either a signal intensity or signal frequency obtained from a detection result in the acoustic sensor 12. The method of calculating the component concentration is disclosed in Patent Literature 1, for example.

The calculation unit 2 and the recording unit 3 described in the present embodiments can be achieved by a computer including a CPU (Central Processing Unit), a storage device, and an interface, as well as a program that controls these hardware resources. FIG. 10 illustrates a configuration example of this computer. The computer includes a CPU 300, a storage device 301, and an interface device (hereinafter abbreviated to I/F) 302. The acoustic sensor 12, the light source 10/10a, the sweep mechanism 16, and the like, for example, are connected to the I/F 302. The program for achieving the optical absorption coefficient measurement method or the component concentration measurement method of embodiments of the present invention in such a computer is stored in the storage device 301. The CPU 300 executes the processing described in the present embodiments in accordance with the program stored in the storage device 301.

### INDUSTRIAL APPLICABILITY

Embodiments of the present invention are applicable to a technology of measuring an optical absorption coefficient distribution or a component concentration distribution of an object to be measured, for example.

### REFERENCE SIGNS LIST

1 Photoacoustic probe
2 Calculation unit
3 Recording unit
10 Light source
10a Array light source
11, 11a Optical system
12 Acoustic sensor
13 Propagation member
14 Reflection member
15 Acoustic matching layer
16 Sweep mechanism
20 Object to be measured
16o Three-axis manipulator
161, 162, 165 Mirror
163 Acousto-optic modulator
164 Converging lens
166 Mirror array device
167 Optical switch
168 Fiber bundle

1-8. (canceled)

9. A photoacoustic probe comprising:
a light source configured to emit one or more light rays;
an acoustic sensor arranged such that an axial direction is parallel with a depth direction of an object to be measured, wherein the acoustic sensor is configured to detect a sound produced from the object to be measured;
a propagation member configured to propagate light from the light source to the object to be measured and to propagate the sound produced from the object to be measured by emission of the light from the light source to the acoustic sensor;
a reflection member provided within the propagation member, wherein the reflection member is configured to reflect the light from the light source and to emit reflected light to the object to be measured in the axial direction of the acoustic sensor; and
a sweep mechanism configured to change a position at which the light from the light source enters the reflection member.

10. The photoacoustic probe according to claim 9, further comprising an acoustic matching layer provided between the propagation member and the object to be measured.

11. The photoacoustic probe according to claim 9, wherein the sweep mechanism comprises a plurality of mirrors configured to be pivotable so as to change the position at which the light from the light source enters the reflection member.

12. The photoacoustic probe according to claim 9, wherein the sweep mechanism comprises:
an acousto-optic modulator configured to deflect the light from the light source; and
a converging lens configured to converge the light from the acousto-optic modulator to cause the light to enter the reflection member.

13. The photoacoustic probe according to claim 9, wherein the light source comprises an array light source obtained by integrating a plurality of light emitting elements.

14. The photoacoustic probe according to claim 13, wherein the sweep mechanism comprises a mirror array device configured to selectively cause specific light rays among a plurality of light rays emitted from the array light source to enter the reflection member.

15. The photoacoustic probe according to claim 13, wherein the sweep mechanism comprises:
an optical switch configured to selectively pass specific light rays among a plurality of light rays emitted from the array light source; and
a fiber bundle configured to cause the light rays passed through the optical switch among the plurality of light rays emitted from the array light source to enter the reflection member.

16. A photoacoustic probe comprising:
a light source configured to emit one or more light rays;
an optical system configured to shape the light rays from the light source;
an acoustic sensor arranged such that an axial direction is parallel with a depth direction of an object to be measured, wherein the acoustic sensor is configured to detect a sound produced from the object to be measured;
a propagation member configured to propagate light from the light source to the object to be measured and to propagate the sound produced from the object to be measured by emission of the light from the light source to the acoustic sensor;
a reflection member provided within the propagation member, wherein the reflection member is configured to reflect the light from the light source and to emit reflected light to the object to be measured in the axial direction of the acoustic sensor; and
a sweep mechanism configured to change a position at which the light from the light source enters the reflection member.

17. The photoacoustic probe according to claim 16, wherein the optical system is configured to set a size of an optical spot to be formed within the object to be measured such that the sound produced from the object to be measured has a frequency of a desired value.

18. The photoacoustic probe according to claim 16, wherein the optical system is configured to shape the light from the light source such that an optical spot to be formed within the object to be measured has a columnar shape whose circular cross section is parallel with the depth direction of the object to be measured.

19. The photoacoustic probe according to claim 16, further comprising an acoustic matching layer provided between the propagation member and the object to be measured.

20. The photoacoustic probe according to claim 16, wherein the sweep mechanism comprises a plurality of mirrors configured to be pivotable so as to change the position at which the light from the light source enters the reflection member.

21. The photoacoustic probe according to claim 16, wherein the sweep mechanism comprises:
an acousto-optic modulator configured to deflect the light from the light source; and
a converging lens configured to converge the light from the acousto-optic modulator to cause the light to enter the reflection member.

22. The photoacoustic probe according to claim 16, wherein the light source comprises an array light source obtained by integrating a plurality of light emitting elements.

23. The photoacoustic probe according to claim 22, wherein the sweep mechanism comprises a mirror array device configured to selectively cause specific light rays among a plurality of light rays emitted from the array light source to enter the reflection member.

24. The photoacoustic probe according to claim 22, wherein the sweep mechanism comprises:
an optical switch configured to selectively pass specific light rays among a plurality of light rays emitted from the array light source; and
a fiber bundle configured to cause the light rays passed through the optical switch among the plurality of light rays emitted from the array light source to enter the reflection member.
